(19) United States
(12) Patent Application Publication
(10) Pub. No.: US 2023/0390742 A1
(43) Pub. Date: Dec. 7, 2023

(54) MIXED CATALYTIC SYSTEM FOR THE CONVERSION OF CO2 AND/OR OF CO IN A COLD PLASMA-CATALYSIS HYBRID PROCESS

(71) Applicants: PARIS SCIENCES ET LETTRES, Paris (FR); CENTRE NATIONAL DE LA RECHERCHE SCIENTIFIQUE, Paris (FR); SORBONNE UNIVERSITE, Paris (FR); ECOLE NATIONALE SUPERIEURE DE CHIMIE DE PARIS, Paris (FR)

(72) Inventors: Maria Elena GALVEZ-PARRUCA, PANTIN (FR); Maria MIKHAIL, PARIS (FR); Stéphanie OGNIER, PARIS (FR); Patrick DA COSTA, PANTIN (FR); Simeon CAVADIAS, Paris (FR); Michael TATOULIAN, Paris (FR)

(21) Appl. No.: 18/034,647

(22) PCT Filed: Oct. 29, 2021
(86) PCT No.: PCT/FR2021/051906

§ 371 Date: Apr. 28, 2023

(30) Foreign Application Priority Data
Oct. 29, 2020 (FR) 2011113

(51) Int. Cl. B01J 23/755; B01J 21/06; B01J 23/10; C07C 1/12; B01J 37/08

(52) U.S. Cl. CPC B01J 23/755; B01J 21/066; B01J 23/10; C07C 1/12; B01J 37/08; B01J 37/16

(57) ABSTRACT

The present invention relates to a catalytic system comprising:—a support comprising cerium and/or zirconium,—nickel, and—a promoter chosen from lanthanides, yttrium, strontium, copper, manganese, cobalt and mixtures thereof, wherein it is not possible for the lanthanide to be cerium. It also relates to a process for preparing such a catalytic system, and also to a process for converting a gas comprising CO2 and/or CO in the presence of such a catalytic system and of a cold plasma, preferentially generated by dielectric barrier discharge (DBD).

### FIELD OF THE INVENTION

The present invention relates to a novel catalytic system capable of being activated by a cold plasma, in particular generated by “dielectric barrier discharge”, called DBD plasma, and the process for preparing said catalytic system. The invention also relates to a process for converting CO₂ and/or CO implementing said catalytic system, for example to produce hydrocarbons, in particular methane, alcohols, carbon monoxide or formic acid.

### PRIOR ART

The reduction of emissions of CO₂ into the atmosphere is currently an important need, because of the exponential increase of these emissions of CO₂ over the last decade, which has led to significant environmental damage such as global warming or the acidification of seawater. Consequently, the technologies that involve the conversion of CO₂ into fuels offer precious advantages for reducing a significant quantity of CO₂ and storing this renewable energy. For this purpose, the carbon dioxide can be transformed in the form of a usable element, typically methane.

Thus, among the various reactions of hydrogenation of CO₂, the most well-known is the Sabatier reaction (1), which allows to convert CO₂ into methane. Methane is a carbon-neutral fuel and allows the storage of renewable electricity outside of peak hours (“power-to-gas” concept).

CO₂ (g)+4H₂ (g)->CH₄ (g)+2H₂O (g), ΔH °25° C.=−165.3 kJ/mol (1)

The Sabatier reaction is exothermic and thermodynamically favourable at low temperature. However, it is seriously hampered by its slow reaction kinetics because of the high stability of CO₂. Thus, the use of catalysts is crucial for obtaining a satisfactory conversion rate.

Ocampo et al. (1) described the use of solid solutions of mixed oxide of cerium and of zirconium for the preparation of nickel catalysts for the methanation of CO₂. The CeO₂—ZrO₂ mixed oxides have good physico-chemical properties, such as a good capacity for storing oxygen and good mobility, an increased surface basicity favouring the adsorption of CO₂, as well as suitable thermal stability.

Cold plasmas, equivalently called “non-thermal plasmas”, in particular the plasmas generated by dielectric barrier discharge (DBD), can be used in association with a catalyst to improve the performance of the methanation of CO₂ (2)-(9). Such a hybrid plasma-catalyst process has several advantages with respect to conventional catalysis, since it operates at atmospheric pressure. This hybrid process can be carried out at low temperature (e.g. 180° C.-240° C.) and allows to obtain better methane selectivity (>95%), without any secondary reaction. However, it remains very costly in terms of energy. The cost of the energy is defined as the quantity of energy consumed by the process per converted molecule, often expressed in kJ or eV per molecule.

The inventors previously demonstrated the efficient association of a catalyst containing mixed oxide of cerium and of zirconium, impregnated with nickel with a cold plasma such as a DBD plasma to carry out the Sabatier reaction at low temperature (e.g. less than 250° C.) (10). However, with this catalyst system, the energy consumption remains significant to obtain a satisfactory rate of conversion of the CO₂.

There is therefore a need for an efficient catalyst for the reaction of conversion of a gas comprising CO₂ and/or carbon monoxide (CO), using a cold plasma-catalysis hybrid process, said catalyst allowing a reduction in the energy consumption in the reaction and/or an improvement of the catalytic performance, namely the rate of conversion of the gas, the selectivity and the catalytic stability, in order to obtain for example hydrocarbons or alcohols.

### SUMMARY OF THE INVENTION

One goal of the invention is therefore to propose a new catalyst, also called catalytic system, allowing to convert CO₂ and/or CO into fuel, including the hydrocarbons, the alcohols, formic acid, carbon monoxide (if the reaction is carried out starting from CO₂) and mixtures thereof, with a reduced energy consumption and a rate of conversion of the CO₂ and/or of the CO improved with respect to those described in the prior art.

A first object of the present invention relates to a catalytic system comprising:
a support comprising cerium and/or zirconium,
nickel, and
a promoter chosen from the lanthanides, yttrium, strontium, copper, manganese, cobalt, iron and mixtures thereof, wherein the lanthanide cannot be cerium.

A second object of the present invention relates to a process for preparing a catalytic system as defined above by placing the support or a precursor thereof in contact with a precursor of nickel and a precursor of the promoter.

A third object of the present invention relates to a process for converting a gas comprising CO₂ and/or CO in the presence of a catalytic system as defined above and a cold plasma.

### DESCRIPTION OF THE DRAWINGS

FIG. 1 illustrates the experimental device used in an example of hydrogenation of CO₂, methanation, by a DBD plasma+ catalysis process according to the invention.

FIG. 2 illustrates the catalytic activity under DBD plasma of catalytic systems according to the invention for the methanation of CO_{2:} A) rate of conversion of the CO₂ obtained according to the energy consumption (power) with a control catalytic system (NiCZ) or a catalytic system according to the invention; B) methane (CH₄) selectivity obtained according to the energy consumption (power) with a control catalytic system (NiCZ) or a catalytic system according to the invention.

FIG. 3 illustrates the catalytic activity under DBD plasma of catalytic systems according to the invention for the methanation of CO_{2:} A) rate of conversion of the CO₂ obtained according to the energy consumption (power) with a control catalytic system (NiCZ) or a catalytic system according to the invention with yttrium at various mass concentrations; B) methane (CH₄) selectivity obtained with respect to CO according to the energy consumption (power) with a control catalytic system or a catalytic system according to the invention with yttrium at various mass concentrations.

### DETAILED DESCRIPTION OF THE INVENTION

### The Catalytic System

The present invention relates to a catalytic system comprising:
a support comprising cerium and/or zirconium,
nickel, and
a promoter chosen from the lanthanides, yttrium, strontium, copper, manganese, cobalt and mixtures thereof, wherein the lanthanide cannot be cerium.

In the context of the present invention, the terms “catalytic system” and “catalyst” are interchangeable.

The support according to the invention is in particular chosen for its capacity to adsorb CO₂ and/or CO at its surface and its thermal stability. “Cerium” and “zirconium” mean in the sense of the present invention cerium and zirconium in all their oxidation states. Preferably, the term “cerium” used to define the support designates CeO₂ cerium oxide and the term “zirconium” designates ZrO₂ zirconium oxide.

The support can thus comprise, in particular consists of, cerium, zirconium or a mixture of cerium and of zirconium. Advantageously, the support comprises, in particular consists of, cerium, optionally in combination with zirconium. More preferably, the support of the catalytic system comprises, in particular consists of, a mixture of cerium and of zirconium.

Advantageously, the support can thus comprise, in particular consists of, a cerium oxide, a zirconium oxide or a mixed oxide of cerium and of zirconium. Advantageously, the support comprises, in particular consists of, cerium oxide, optionally in combination with zirconium oxide. More preferably, the support of the catalytic system comprises, in particular consists of, a mixed oxide of cerium and of zirconium (abbreviated CeO₂—ZrO₂ or CeZr mixed oxide or CZ).

When the support is a mixture of cerium and of zirconium, in particular a CeZr mixed oxide, the cerium/zirconium molar ratio (Ce/Zr) is advantageously within a range going from 90/10 to 40/60, preferably from to 50/50, in particular 70/30 to 50/50. Preferably, the cerium/zirconium ratio is approximately 60/40. The Ce/Zr ratio in particular modulates the catalytic performance of the system. Indeed, the adsorption of the reactants at the surface of the support is generally favoured by the presence of inherent defects present on said surface, mainly due to a lack of oxygen. The addition of Zr4+ ions into the network of the cerium oxide during the formation of the CeZr mixed oxide allows a higher number of defects and favours the mobility of the oxygen at the surface of the support thus leading to increased catalytic performance.

“Approximately” means in the present description that the value in question can be lower or higher by 10%, in particular 5%, in particular 1%, than the indicated value. The nickel corresponds to the active phase of the catalytic system. “Nickel” means in the sense of the present invention nickel in all its oxidation states. This can therefore be nickel in its 0 oxidation state, that is to say in metallic form, in its II oxidation state (e.g. in the form of nickel (II) oxide (NiO)) or in its III oxidation state (e.g. in the form of nickel (III) oxide (Ni₂O₃)). The mass concentration of nickel in the catalytic system advantageously ranges from 3% to 30% by weight relative to the weight of the support, preferably from 5% to 20%, even more preferably from 7% to 17%. In a particularly advantageous manner, the catalytic system comprises approximately 15% by weight of nickel relative to the weight of the support.

The catalytic system according to the invention allows to improve the energy performance during the conversion, in particular the hydrogenation, of the gas comprising CO₂ and/or CO in the presence of a non-thermal plasma with a reduction of the power consumed, and preferably while improving the catalytic performance of the reaction, that is to say the rate of conversion of the CO₂ and/or of the CO and/or the selectivity towards the desired product.

For this purpose, the catalytic system comprises a promoter that acts as a dopant and that in particular modifies the conductive properties of the catalytic system. The promoter according to the invention advantageously has suitable physico-chemical surface properties to assist in fixing the CO₂ and/or the CO and a dielectric constant that allows to improve the conductivity of the resulting catalytic system. “Suitable physico-chemical surface properties” means for example a large number of surface defects, a suitable basicity, an oxygen gap and/or an ionic radius suitable for incorporation into the crystalline network of the cerium oxide and favouring redox cycles.

Thus, in the context of the present invention, the promoter is chosen from the lanthanides, yttrium, strontium, copper, manganese, cobalt, iron and mixtures thereof, wherein the lanthanide cannot be cerium. In the context of the present invention, the promoter is in any of its oxidation states and in particular in metallic form or in the form of oxide.

The lanthanides form a family of chemical elements including lanthanum, cerium, praseodymium, neodymium, promethium, samarium, europium, gadolinium, terbium, dysprosium, holmium, erbium, thulium, ytterbium and lutecium. In the context of the present invention, the lanthanide cannot be cerium, the latter already being potentially present in the support. Preferably, the lanthanide is gadolinium or lanthanum, more preferably gadolinium.

The promoter is advantageously chosen from the lanthanides, yttrium and mixtures thereof. Preferably, the promoter is chosen from gadolinium, lanthanum, yttrium and mixtures thereof. More preferably, the promoter is chosen from gadolinium, yttrium and mixtures thereof, and even more preferably from gadolinium and yttrium.

The mass concentration of promoter in the catalytic system varies from 0.1% to 20% by weight relative to the weight of the support, for example from 0.2 to 20% by weight, in particular from 0.5% to 15% by weight, preferably from 1% to 10% by weight, in particular from 2% to 8% by weight, even more preferably from 4 to 7% by weight.

The quantity of nickel and/or of promoter can be adjusted according to the nature of the promoter used and/or the type of conversion reaction implemented, that is to say according to the type of product generated by the conversion, in particular hydrogenation, reaction.

Thus, according to one embodiment, when the promoter is gadolinium, its mass concentration in the catalytic system is preferably approximately 4% by weight relative to the weight of the support. According to another embodiment, when the promoter is yttrium, its mass concentration in the catalytic system is preferably approximately 7% by weight relative to the weight of the support.

The catalytic system is advantageously in the form of powder. Preferably, the grains forming the powder have an average size between 1 μm and 1 mm, for example between 100 μm and 1 cm, in particular between 200 μm and 800 μm, preferably approximately 500 μm. The size of the grains can in particular be adapted according to the scale of production used. The catalytic system can also be in the form of balls (in particular by compression of the powder in a mould) having an average size between 1 mm and 5 cm. The determination of the average size of the grains and of the balls can be carried out by laser granulometry.

Advantageously, the support, the nickel and the promoter form a homogenous mixture. This means that the nickel and the promoter are uniformly distributed in the totality of the volume of the catalyst.

### The Process for Preparing the Catalytic System

The present invention also relates to a process for preparing a catalytic system as defined above comprising a step of placing the support or a precursor thereof in contact with a precursor of nickel and a precursor of the promoter, optionally followed by a calcination step, which is optionally followed by a reduction step.

The step of placing the support in contact with a precursor of nickel and a precursor of the promoter allows to form a solid comprising the support, the nickel and the promoter. Preferably, this solid is then calcined. Such a calcination step can be carried out for example at a temperature between 300 and 600° C. This calcination step can be carried out for example for 3 h or more, in particular between 3 h and 6 h. The calcination step can also be carried out in situ in the non-thermal plasma device, for example for a duration of 3 h or more, preferably between 3 h and 5 h, at a temperature between 300° C. and 600° C. The calcination step leads to the oxidation of the various components of the catalytic system (support, nickel and promoter).

The calcination step can optionally be followed by a reduction step so as to change the oxidation state of the nickel and of the promoter (to have nickel and metallic promoter), and optionally that of the support. The reduction can be total or partial, that is to say that only a part of the components of the catalytic system can be reduced. The reduction step can be carried out in situ in the non-thermal plasma device under hydrogen.

Several (for example 2) sequences comprising a step of placing in contact and a calcination step can be carried out successively, wherein the conditions of the step of placing in contact and of the calcination step can be different from one sequence to another. The second step of placing in contact or any other later step of placing in contact can be a step of placing the support in contact only with a precursor of nickel or a precursor of promoter.

The calcined solid obtained is then advantageously transformed into a powder, in particular by grinding and optionally screening. Preferably, the grains forming the powder have an average size between 1 μm and 1 mm, for example between 100 μm and 1 mm, in particular between 200 μm and 800 μm, preferably of approximately 500 μm. The catalytic system can also be in the form of balls having an average size between 1 mm and 5 cm. The determination of the average size of the grains or of the balls can be carried out by laser granulometry.

The step of placing the support in contact with a precursor of nickel and a precursor of the promoter can be carried out for example by impregnation, by coprecipitation or by sol-gel reaction.

The precursor of nickel can be any chemical compound, or mixture of chemical compounds, containing nickel and more particularly can be a nickel salt, a nickel oxide or a mixture thereof, preferably a nickel salt or a mixture of nickel salts. The nickel salt can be for example chosen from the chloride, the nitrate, the sulphate, the carbonate, the acetate, the acetylacetonate, the tartrate, and the citrate of nickel and mixtures thereof, preferably is nickel nitrate, in particular hexahydrate. The nickel oxide can be an oxide of nickel (II) or (III), respectively noted as NiO and Ni₂O₃.

The precursor of the promoter can be any chemical compound, or mixture of chemical compounds, containing the metal used as a promoter and more particularly can be a salt of said metal, an oxide of said metal or a mixture thereof, preferably a salt of said metal or a mixture of salts of said metal. The salt of said metal can be for example chosen from the chloride, the nitrate, the sulphate, the carbonate, the acetate, the acetylacetonate, the tartrate, and the citrate of said metal and mixtures thereof, preferably the nitrate of said metal. In the case in which the promoter is a mixture of several metals, the precursor can be a mixture of various types of salts and/or of oxides of these metals.

In the context of the present invention, the term “salt” designates a non-hydrated salt or a hydrated, or even multi-hydrated, salt.

According to a first embodiment, the step of placing the support in contact with a precursor of nickel and a precursor of the promoter is carried out by impregnation and comprises the following steps:
a) preparation of an aqueous solution comprising a precursor of nickel and a precursor of the promoter,
b) addition of the support to the aqueous solution resulting from step a) to give a suspension,
c) mixture of the suspension resulting from step b),
d) recovery of the solid comprising the support, the nickel and the promoter obtained in step b).

This process is also called by wet impregnation. It involves impregnating the support with the precursor of nickel and the precursor of the promoter.

During step a), a suitable mass of the precursor of nickel and a suitable mass of the precursor of the promoter are added to a suitable volume of water. The addition of these precursors is carried out in particular at ambient temperature, that is to say a temperature of 15 to 40° C., advantageously of 20 to 30° C., in particular of 20 to 25° C. “Suitable mass” and “suitable volume” mean the quantities of precursors and of water suitable for obtaining the desired mass concentrations of nickel and of promoter in the final catalytic system.

During step b), a suitable mass of support is added to the solution resulting from step a). “Suitable mass” means a quantity of support suitable for obtaining the desired mass concentrations of nickel and of promoter in the final catalytic system.

During step c), the suspension resulting from step b) is mixed, in particular by stirring, in particular at ambient temperature. This mixing step can be carried out advantageously for a duration of 30 min or more, in particular of 30 min to several hours, for example of 30 min to 3 h.

Step d) can be carried out by elimination of the excess water, in particular by evaporation of the water or filtration, then drying of the resulting solid. The drying step can be carried out for example at a temperature greater than or equal to 70° C., for example between 70° C. and 150° C., typically at a temperature of approximately 100° C. This drying can be carried out for 7 h or more, typically for a duration ranging from 7 h to 48 h.

According to a second embodiment, the step of placing the support in contact with a precursor of nickel and a precursor of the promoter is carried out by coprecipitation and comprises the following steps:
a′) preparation of an aqueous solution comprising a precursor of nickel and a precursor of the promoter,
b′) addition of the support to the aqueous solution resulting from step a′) to give a suspension,
c′) addition of a base to the suspension resulting from step b′) until the pH of the suspension is between 8 and 12, preferably until the pH is approximately 10,
d′) mixture of the suspension resulting from step c′),
e′) recovery of the solid comprising the support, the nickel and the promoter obtained in step d′).

This process allows to precipitate nickel hydroxide and hydroxide of the metal used as a promoter at the surface of the support.

The steps a′) and b′) are carried out in the same conditions as steps a) and b) respectively mentioned above.

During step c′), a base is added to the suspension resulting from step b′). This base is advantageously a hydroxide salt such as the hydroxide of sodium or of potassium or the carbonate of sodium or of potassium in particular sodium hydroxide. It can be used in the form of a solution, in particular aqueous.

The base is added until the pH of the suspension is 10 or more, in particular approximately 10. This addition is progressive, in particular drop by drop. Since the suspension consists of a solid in suspension in a liquid solution, “pH of the suspension” means the pH of the liquid solution of the suspension.

Step c′) is advantageously carried out at a temperature between 60° C. and 100° C., in particular between ° C. and 90° C., preferably equal to approximately 80° C.

Step d′) aims to precipitate the nickel hydroxide and the hydroxide of the metal used as a promoter at the surface of the support. The mixing, in particular by stirring, is advantageously carried out at a temperature between 60° C. and 100° C., in particular between 70° C. and ° C., preferably equal to approximately 80° C. This mixing step can be carried out for a duration of 2 h or more, preferably between 2 h and 5 h, typically for approximately 3 h.

Step e′) can be carried out like the step d) mentioned above.

According to a third embodiment, the step of placing the support in contact with a precursor of nickel and a precursor of the promoter is carried out by sol-gel reaction and comprises the following steps:
a″) preparation of a solution comprising a precursor of nickel, a precursor of the promoter and a support precursor in propionic acid,
b″) reflux heating of the solution of step a″),
c″) elimination of the excess propionic acid and recovery of the catalytic system.

In step a″), the support precursor corresponds to a precursor of cerium, a precursor of zirconium or a mixture of precursor of cerium and of precursor of zirconium according to whether the support is cerium, zirconium or a mixture of cerium and of zirconium. In the case in which the support is a mixture, in particular a mixed oxide, of cerium and of zirconium, a solution of precursor of cerium and a solution of precursor of zirconium are advantageously prepared separately then mixed in a ratio allowing to obtain the desired Ce/Zr molar ratio in the final mixture, in particular the mixed oxide, of cerium and of zirconium.

The precursor of cerium or of zirconium can be more particularly a salt of cerium or of zirconium or a mixture of salts of cerium or of zirconium. The salt of cerium or of zirconium can for example be chosen from the chloride, the nitrate, the sulphate, the carbonate, the acetate, the acetylacetonate, the tartrate, and the citrate of cerium or of zirconium and mixtures thereof. Preferably, the precursor of cerium is cerium acetate, in particular sesquihydrate, and the precursor of zirconium is zirconium acetylacetonate.

In order to prepare the desired quantity of catalyst, the precursor of the support, as well as the precursors of nickel (e.g. the nickel (II) acetate tetrahydrate) and of the promoter are advantageously dissolved separately in propionic acid, in particular with heating (e.g. to 80-120° C., in particular approximately 100° C., advantageously for 30 min or more, for example for approximately 1 h).

The step b″) of reflux heating is carried out for a time sufficient to obtain propionates. For example, the solution is heated for a time between 1 h and 3 h.

Step c″) can be carried out by evaporation of the propionic acid. If the product resulting from step c″) is a gel, it can be solidified before carrying out an optional step of calcination, in particular using liquid nitrogen, so that the product resulting from step c″) is a solid.

The support used in the process for preparing the catalytic system can be a commercially available support.

According to a specific embodiment, when the step of placing the support in contact with a precursor of nickel and a precursor of the promoter is carried out by impregnation or by coprecipitation, the process for preparing the catalytic system comprises the previous preparation of the support according to the following steps:
i) preparation of a solution of support precursor in propionic acid,
ii) reflux heating of the solution from step i),
iii) elimination of the excess propionic acid, and
iv) calcination of the solid resulting from step iii) to give the support.

In step i), the support precursor corresponds to a precursor of cerium, a precursor of zirconium or a mixture of precursor of cerium and of precursor of zirconium as defined above for step a″) of the process for preparing the catalytic system by sol-gel reaction. In the case in which the support is a mixture, in particular a mixed oxide, of cerium and of zirconium, a solution of precursor of cerium and a solution of precursor of zirconium are advantageously prepared separately then mixed in a ratio allowing to obtain the desired Ce/Zr molar ratio in the final mixture, in particular the mixed oxide, of cerium and of zirconium.

The step ii) of reflux heating is carried out for a time sufficient to obtain propionates. For example, the solution is heated for a time between 1 h and 3 h.

Step iii) can be carried out by evaporation of the propionic acid. If the product resulting from step iii) is a gel, it can be solidified before carrying out step iv), in particular using liquid nitrogen, so that the product resulting from step iii) is a solid.

The product resulting from step iii) is then calcined, for example at a temperature between 300° C. and 600° C. This calcination step can be carried out for 3 h or more, for example for a duration ranging from 3 h to 6 h. This step leads to the thermal decomposition of the propionates in order to obtain oxides.

### The Process for Converting CO₂ and/or CO

The present invention also relates to a process for converting a gas comprising CO₂ and/or CO in the presence of a catalytic system as defined above and a cold plasma, advantageously a plasma generated by dielectric barrier discharge also called dielectric barrier discharge plasma or DBD plasma.

The process for converting a gas comprising CO₂ and/or CO according to the invention involves the conversion of the CO₂ and/or of the CO contained in this gas, in particular in the presence of dihydrogen, which involves the reduction and in particular the hydrogenation of the CO₂ and/or of the CO. This process thus allows in particular to generate:
one or more hydrocarbons (e.g. methane), one or more alcohols (e.g. methanol, ethanol), carbon monoxide, formic acid and/or mixtures thereof from the conversion of the CO₂, and/or
one or more hydrocarbons (e.g. methane), one or more alcohols (e.g. methanol, ethanol), formic acid and/or mixtures thereof from the conversion of the CO.

The process for converting a gas comprising CO₂ and/or CO according to the invention corresponds in particular to a hydrogenation of the CO₂ and/or of the CO respectively into one or more hydrocarbons (e.g. methane), one or more alcohols (e.g. methanol, ethanol), formic acid and/or a mixture thereof.

When a hydrocarbon is generated, it is preferably methane, ethane, propane, butane, pentane, hexane or mixtures thereof, more preferably methane.

When an alcohol is generated, it is preferably methanol, ethanol, propanol, butanol or mixtures thereof, more preferably methanol or ethanol.

Preferably, the conversion, in particular the hydrogenation, of the CO₂ and/or of the CO according to the process of the invention generates methane or methanol, more preferably methane. The product obtained depends in particular on the molar ratio between the CO₂ and/or the CO and the dihydrogen used to carry out the conversion reaction, a ratio that a person skilled in the art is perfectly capable of determining according to the desired product. For example, to obtain methane from CO₂, the CO₂/H₂ ratio is 1:4. To obtain methanol from CO₂, the CO₂/H₂ ratio is 1:3. To obtain CO from CO₂, the CO₂/H₂ ratio is 1:1.

The process for converting a gas comprising CO₂ and/or CO according to the invention comprises the placement of the catalytic system according to the invention in a device adapted to the generation of a cold plasma, in particular a DBD plasma. Such a device advantageously comprises a reactor in which the catalytic system is placed as well as an electric system allowing the generation of a cold plasma, in particular a DBD plasma. It can also comprise a system allowing to analyse the products generated by the reaction of conversion of the CO₂ and/or of the CO.

The walls of the reactor can be made of a metal material or, in the case in which a DBD plasma is used, of a dielectric material such as quartz or a ceramic. The reactor can be advantageously in a cylindrical shape. It comprises in particular at least one inlet allowing its supply with gas comprising CO₂ and/or CO, and H₂ in the form of gas, and an outlet for evacuating the products formed, advantageously in the form of gas. The inlet of the reactor is advantageously connected to a first container intended to contain the gas comprising CO₂ and/or CO and to a second container intended to contain the H₂. A system for mixing the gases (gas comprising CO₂ and/or CO and H₂), in particular in a given ratio of CO₂ and/or CO/H₂, can be present between the containers intended to respectively receive the gas comprising CO₂ and/or CO and the H₂ and the inlet of the reactor. The outlet of the reactor is advantageously connected to a container intended to contain the products generated by the conversion reaction. A cooling system can be present between the container intended to receive the products generated by the conversion reaction and the outlet of the reactor so as to condense and eliminate the water that could be formed during the conversion reaction.

The electric system allowing the generation of DBD plasma advantageously comprises two electrodes, for example one placed in the reactor and the other placed outside the reactor, for example around a part of the cylinder when the reactor is cylindrical, between which a voltage can be applied so as to generate a DBD plasma. The catalytic system is thus placed inside the reactor, between these two electrodes. It constitutes the catalytic bed. The catalytic system according to the invention is activated by the creation in the catalytic system (more particularly between the grains of said catalytic system) or nearby (upstream or downstream of the catalytic system, in the flow of gas) of a strong electric field, typically between 10⁵ and 10¹⁰ V/m, which can vary over time. This strong electric field allows the ionisation of a part of the gas and the excitation of atoms and of molecules present in the gaseous phase, typically the molecules of CO and/or of CO₂ and of H₂, by the electrons thus stripped and accelerated. While the temperature of the electrons is several thousand or several tens of thousands kelvins, the majority of the gas remains at ambient temperature or at several hundred kelvins. This is why this is called a “cold” or “non-thermal” (which is not at thermodynamic equilibrium) plasma.

According to the speed of variation of this strong electric field, the plasma can be qualified as radiofrequency, microwave (according to the frequencies corresponding to these electromagnetic ranges), or more advantageously low-frequency (50 Hz<f<1 MHz) or pulsed (pulses having durations between fns and 1 ms, with fast rise times of 0.1 ns to 100 μs).

In the two latter cases, two electrodes are in particular placed on either side of the catalyst and a generator of high voltage that creates a difference in potential between these electrodes in order to create the desired electric field, whether it is alternating, low-frequency or pulsed. Advantageously, if the reactor has a cylindrical shape, an electrode can be placed inside the reactor and the other outside the reactor. The catalytic system is then placed inside the reactor, between these two electrodes. It constitutes the catalytic bed.

In the specific case of a DBD plasma, at least one layer of dielectric material, preferably two, are inserted between the two electrodes. These dielectric materials can also be the walls of the catalytic reactor or not.

In addition to the creation of a cold plasma state, the strong electric field created is responsible for the negative or positive polarisation of the catalytic sites. This polarisation induces reactions of adsorption and of desorption even at low temperature (for example at temperatures below 300° C., or even below 200° C.). Without polarisation (conventional process), the working temperature is higher, generally from 300° C. to 420° C.

In an alternative of the invention, the catalytic system of the invention is activated by a DBD plasma by providing an electric power of 0.001 W/g to 3 W/g of catalytic system (namely the catalytic system present in the reactor, that is to say in the catalytic bed).

Before the step of conversion, in particular of hydrogenation, the catalytic system according to the invention, in the case in which its components are in the form of oxides, is advantageously reduced in situ under cold plasma, preferably a DBD plasma, under H₂ as the discharge gas.

The conversion reaction is advantageously carried out at a pressure greater than or equal to the atmospheric pressure (10⁵ Pa), for example at a pressure ranging from the atmospheric pressure to 3.10⁵ Pa. The conversion reaction can be carried out in pseudo-adiabatic conditions, that is to say without thermal insulation and without external heating, or in isothermal conditions, that is to say with thermal insulation.

In the case of a DBD plasma, the latter is advantageously generated between the electrodes of the electric system allowing to generate the DBD plasma by applying a voltage between the two electrodes, between 1 and 25 kV, preferably between 8 and 16 kV, in particular with a frequency between 1 kHz and 100 kHz.

The temperature of the catalytic bed depends on the voltage applied. Typically this temperature is less than 300° C., in particular it is between 150° C. and 270° C.

The gas hourly space velocity (GHSV) is in particular between 1000 h⁻¹ and 100000 h⁻¹, preferably between 30000 h⁻¹ and 50000 h ⁻¹.

During the reaction of conversion, in particular of hydrogenation, of the gas comprising CO₂ and/or CO via a cold plasma-catalysis hybrid process by using the catalytic system according to the invention, the observed rate of conversion of the CO₂ and/or of the CO is typically greater than 70% with a consumed power lower than 5 W, and even greater than 80% with a consumed power lower than 8 W. The selectivity towards the desired product (for example methane if the conversion reaction is a methanation reaction) is typically greater than 95%, and even greater than 99%.

### EXAMPLES

### A) Materials and processes

### I. Preparation of the catalytic systems

### Supports

Several supports were used:
1) A commercial cerium-zirconium mixed oxide (CeₓZr₁₋ₓO₂, x=0.58, Solvay);
2) A commercial CeO₂ cerium oxide (Sigma-Aldrich);
3) A commercial ZrO₂ zirconium oxide (Daiichi Kigenso kagaku Kogyo Co);
4) A cerium-zirconium mixed oxide having the molar composition Ce_{0.58}Zr_{0.42}O₂, synthesised by the inventors according to the process described below. This support has the same composition as the support 1).

### Synthesis of the Support 4) of the Type Cerium-Zirconium Mixed Oxide Having the Molar Composition Ce_{0.58}Zr_{0.42}O₂(CZ)

For the synthesis of the support of the CZ type, the starting organometallic salts that were used are cerium (III) acetate sesquihydrate and zirconium (IV) acetylacetonate. These salts are dissolved separately in hot propionic acid for 1 h, so as to obtain solutions having a concentration of 0.12 mol·L⁻¹. It is indispensable to dissolve the suitable starting salts, which will lead to obtaining exclusively the desired metal propionates. The various solutions are then mixed and heated under reflux in a round-bottom flask equipped with a bulb condenser for 90 min (B.P. propionic acid =141° C.), creating mixed propionates. The solvent is then evaporated via a controlled distillation under vacuum until a mixed gel, containing the metal elements in the chosen stoichiometry, is obtained by oligomerisation. This gel is solidified via liquid nitrogen and calcined under air for 6 h, at a temperature of 400 to 600° C., generally at 500° C., by using a temperature ramp of 2 ° C./min. This step leads to the thermal decomposition of the propionates in order to obtain the mixed oxide having the desired composition.

### Synthesis of the Catalytic Systems

Catalytic systems were synthesised by wet impregnation or by coprecipitation.

The catalytic systems were prepared from the three supports described above (cerium oxide, zirconium oxide or cerium and zirconium mixed oxide) with 15% by weight of nickel relative to the weight of the support. The following promoters were investigated, for some with various mass concentrations in the catalytic system: gadolinium (Gd), yttrium (Y), strontium (Sr), lanthanum (La), praseodymium (Pr), manganese (Mn), cobalt (Co), magnesium (Mg), zinc (Zn), potassium (K), indium (In), iron (Fe), sodium (Na) and copper (Cu).

Thus, various natures of support, various processes for preparing the catalytic system, various promoters and various quantities of promoters were studied.

### a) Preparation of the Catalytic System by Wet Impregnation

The catalytic systems with nickel doped by the promoter (metal M) were prepared by the process of wet co-impregnation using an aqueous solution of Ni(NO₃)₂·6H₂O (Sigma-Aldrich) and a precursor of promoter chosen from: Cu(NO₃)₂·5H₂O, Co(NO₃)₂·6H₂O, Mn(NO₃)₂·4H₂O, La(NO₃)₂·6H₂O, Y(NO₃)₂·6H₂O, Sr(NO₃)₂·4H₂O, Gd(NO₃)₂·6H₂O, Mg(NO₃)₂·6H₂O, Zn(NO₃)₂·6H₂O, NaNO₃, KNO₃, and In(NO₃)₂·6H₂O (all commercial, Sigma-Aldrich), according to the desired promoter.

The concentration of nickel is 15% by weight relative to the weight of the support and that of the promoter M varies between 2, 4, 7, 10, 15 and 20% by weight relative to the support. A catalyst without promoter was also prepared as a reference material.

The suitable mass of nickel salt and that of the precursor of promoter are dissolved in a volume of water of 250 mL, at ambient temperature and with stirring. The suitable mass of support is added to the aqueous solution containing the mixture of the metal salts and maintained under stirring for 2 hours. For example, 3.72 g of nickel nitrate (concentration 15% by weight), 1.5 g of yttrium nitrate (concentration 7% by weight) and 5 g of support are mixed according to the preceding procedure. The mixture is then placed in a rotary evaporator for 2 h at 60° C., in order to eliminate the excess water.

After impregnation and evaporation of the water, all the samples were collected, dried in an oven at 100° C. for then calcined under air at 550° C. for 4 h with a temperature ramp of 10 ° C./min. After calcination, the samples of catalytic system were ground by hand and screened to an average grain size between 10 and 50 μm. After this step, several catalysts of various particle sizes were then manufactured according to the following procedure. First of all, the original catalytic powders were pressed into a pellet using a mould, then the pellet was ground into small pieces. Finally, the particles of catalyst were separated into various sizes using several meshes that vary between 30 μm and 5 mm. The best catalytic and energetic performance was obtained for a particle size of approximately 500 μm. The distribution of the size of the grains was certified by laser granulometry.

### b) Preparation of the Catalytic System by Coprecipitation

The catalytic system prepared by coprecipitation comprises a support of commercial cerium and zirconium mixed oxide in the form of powder (CeₓZr₁₋ₓO₂, x=0.58, Solvay) designated by the name CZ.

The catalytic systems of the NiCZ-M type (M being the promoter) were prepared with a mass concentration of Ni of 15% relative to the support. For comparison, a catalyst of the NiCZ type (without promoter) with 15% by weight of nickel relative to the weight of the support was also prepared by the same process.

The powder of CZ was first placed in suspension in the aqueous solution containing a suitable quantity of nickel nitrate and of nitrate of the desired promoter at ambient temperature. In order to synthesise the desired quantity of catalyst, a solution of NaOH at 2M was also prepared. The latter solution was added drop by drop onto the mixture containing the powder of CZ with the nitrates of nickel and of promoter, at a temperature of 80° C. and with stirring, until the value of the pH of the mixed solution reached 10. Then, this mixture was aged for 3 h with vigorous stirring at 80° C., during which the hydroxide of nickel and of promoter was exclusively precipitated at the surface of the support. The pH and the temperature are controlled throughout the synthesis with a sealed pH and temperature tester. The solid thus obtained is then filtered and washed with distilled water and dried in an oven at 100° C. for one night. The solid thus obtained is calcined under air at 550° C. for 4 h with a temperature ramp of 10° C./min. After calcination, the samples of catalytic system were ground by hand and screened to an average grain size between 10 and 50 μm. After this step, several catalysts of various particle sizes were then manufactured according to the procedure described in detail in paragraph a).

### c) Preparation of the Catalytic System by Sequenced Impregnation

The catalytic system is prepared by two successive sequences of impregnation. In other words, a first wet impregnation of the support with the nickel and the desired promoter is carried out according to the process a) above until the calcination step. After the calcination, a new impregnation only with the promoter this time is carried out on the catalytic system coming from the first impregnation.

### II. Experimental Conditions and Parameters of the Hydrogenation of the CO₂: Example of the Methanation of the CO₂

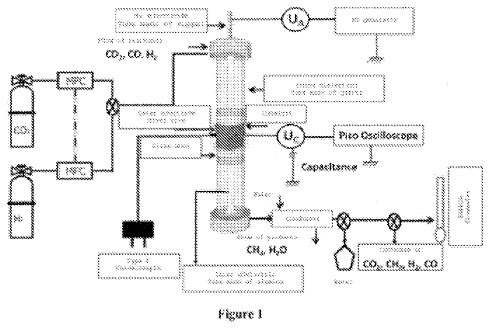

The activity and the selectivity of the catalytic systems with and without promoter in the methanation of the CO₂, in DBD plasma conditions, were evaluated in an experimental device comprising a tubular cylindrical reactor made of quartz, a DBD plasma generator, and the corresponding devices for the supply and the analysis of the gases. A diagram of the DBD plasma device is provided in FIG. 1.

A non-thermal dielectric barrier discharge (DBD) plasma was created between two electrodes: a cylindrical electrode made of copper placed inside a tube made of alumina (3 mm in diameter), surrounded by a coaxial tube made of quartz (inner diameter of 10 mm, thickness of 1 mm), and a steel wire wound around the outer surface of the tube made of quartz, acting as a ground electrode (grounded via an external capacitor of 2nF). In this configuration, a discharge was maintained in a space of 2.5 mm, covering a length of approximately 6.5 mm. Before each methanation experiment, the tubular adiabatic reactor is loaded with 300 mg of catalytic system (grain size of approximately 30 μm) forming the catalytic bed. On both sides of the catalytic bed, glass wool was used to maintain the catalytic system fastened in the discharge zone. Before the catalytic trial, the catalytic systems as synthesised were reduced in situ under non-thermal DBD plasma under H₂ as the discharge gas (voltage=15.0 kV, frequency=70.3 kHz, flow rate=160 ml·min⁻¹ STP) for a duration of 60 minutes. Then, in the conditions of the DBD plasma, the methanation of the CO₂ was carried out at ambient pressure (P=1 atm), in pseudo-adiabatic conditions, with a flow rate of 200 mL/min of a gaseous mixture containing CO₂ and H₂ (H₂:CO_{2=4:1}). These flow conditions correspond to a gas hourly space velocity (GHSV) equal to 44000 h⁻¹.

The plasma-catalysis experiments were carried out at a frequency of 70 kHz, and at voltages between 13 and 16 kV. The voltage applied was measured using an oscilloscope for PC (5000 Series Picoscope, Pico Technology), with a probe (ELDITEST GE 3830). The Lissajous method (11)-(12) was used for the determination of the power of the input plasma. All the experiments were carried out in pseudo-adiabatic conditions (without thermal insulation) and without outside heating (neither the reactor nor the inlet of the gas is heated). The temperature, measured with a Type K thermocouple located on the ground electrode near the catalytic bed, depends strongly on the voltage applied. The liquid water formed by the methanation reaction is eliminated in a condenser. The flow rate of gas exiting this condenser and entering the gas analysis apparatus is regularly measured using a bubble flowmeter. The concentrations of CO₂, H_{2,} CH₄ and CO in the gas exiting the condenser were measured using a gas chromatograph (IGC A20 ML, Delsi Intersmat) equipped with a Carboxen column and a thermal conductivity detector (TCD).

The following equations were used for the calculation of the conversion of the CO₂, of the CH₄ selectivity and of the CO selectivity, all in percentage:

$${X}_{{CO}_{2}} ( \% ) = \frac{{F}_{{CO}_{2}}^{in} - {F}_{{CO}_{2}}^{out}}{{F}_{{CO}_{2}}^{in}} \times 100 \\ {S}_{{CH}_{4}} ( \% ) = \frac{{F}_{{CH}_{4}}^{out}}{{F}_{{CO}_{2}}^{in} - {F}_{{CO}_{2}}^{out}} \times 100 \\ {S}_{CO} ( \% ) = \frac{{F}_{CO}^{out}}{{F}_{{CO}_{2}}^{in} - {F}_{{CO}_{2}}^{out}} \times 100$$

where Fⁱⁿ and F^{out} respectively designate the inlet and outlet flow rate (mol/s) of the species considered at the inlet and at the outlet of the reactor.

### B) Results

The catalytic performance and the energy consumption were evaluated for each catalytic system synthesised in the reaction of methanation of the CO₂ using a DBD plasma according to the process described above.

### I. Nature of the Promoter

Catalysts containing a support of CeZr mixed oxide with 15% by weight of nickel relative to the weight of the support and various promoters were tested. The mass concentration of promoter in the catalytic system is 4% by weight relative to the weight of the support.

The results of their catalytic performance and of the energy consumption according to the nature of the promoter introduced into the catalytic system are summarised in table 1 below. The catalysts of inputs 1, 2, 3, 4, 5, 10, 11, 13 and 14 correspond to the present invention.

TABLE 1

| | | | Rate of | | |
|---|---|---|---|---|---|
| | | | conversion | Selectivity | |
| | | | of the | towards | |
| | Pro- | | CO₂ | methane | Energy |
| Input | moter | Catalyst | (%) | (%) | performance |
| 0 (control) | — | NiCeZr | − | ++ | − |
| | | (reference) | 48 ± 3 | 93 ± 1 | |
| 1 | Y | NiCeZrY | +++ | +++ | +++ |
| | | | 76 ± 3 | 97 ± 1 | |
| 2 | Gd | NiCeZrGd | +++ | +++ | +++ |
| | | | 83 ± 3 | 98 ± 1 | |
| 3 | Co | NiCeZrCo | ++ | ++ | +++ |
| | | | 74 ± 3 | 94 ± 1 | |
| 4 | Sr | NiCeZrSr | ++ | ++ | ++ |
| | | | 71 ± 3 | 91 ± 1 | |
| 5 | Mn | NiCeZrMn | ++ | ++ | ++ |
| | | | 63 ± 3 | 95 ± 1 | |
| 6 | Mg | NiCeZrMg | + | ++ | − |
| | | | 55 ± 3 | 92 ± 1 | |
| 7 | Zn | NiCeZrZn | − | − | − |
| | | | 32 ± 3 | | |
| 8 | K | NiCeZrK | − | − | − |
| | | | 19 ± 3 | | |
| 9 | In | NiCeZrIn | − | − | − |
| | | | 17 ± 3 | | |
| 10 | Pr | NiCeZrPr | ++ | +++ | ++ |
| | | | 72 ± 3 | 96 ± 1 | |
| 11 | Fe | NiCeZrFe | + | ++ | + |
| | | | 53 ± 3 | 94 ± 1 | |
| 12 | Na | NiCeZrNa | − | − | − |
| | | | 18 ± 3 | | |
| 13 | La | NiCeZrLa | + | ++ | ++ |
| | | | 58 ± 3 | 94 ± 1 | |
| 14 | Cu | NiCeZrCu | + | ++ | + |
| | | | 53 ± 3 | 94 ± 1 | |

The catalytic performance is in particular evaluated according to the conversion rate at a reference power of 8 W. A +++ catalytic performance corresponds to a rate of conversion of the CO₂ greater than or equal to 75%. A ++ catalytic performance corresponds to a rate of conversion of the CO₂ greater than or equal to 60%. A + catalytic performance corresponds to a rate of conversion of the CO₂ greater than or equal to 50%. A − catalytic performance corresponds to a conversion rate lower than 50%.

A +++ methane selectivity corresponds to a methane selectivity greater than or equal to 95%. A ++ methane selectivity corresponds to a methane selectivity greater than or equal to 90%. A − methane selectivity corresponds to a methane selectivity lower than 90%.

The energy performance is evaluated according to the power consumed to obtain a conversion rate of at least 60%. A +++ energy performance corresponds to a consumed power less than or equal to 6 W. A ++ energy performance corresponds to a consumed power less than or equal to 9 W. A + energy performance corresponds to a consumed power less than or equal to 12 W. A − energy performance corresponds to a consumed power greater than 12 W.

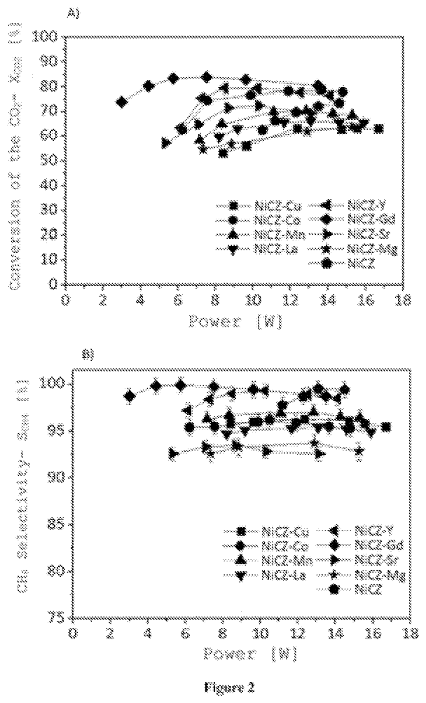

The conversion rate obtained according to the power consumed is shown for each catalytic system according to the invention in FIG. 2 (A). As for FIG. 2 (B), it shows the selectivity towards methane according to the power consumed for each catalyst according to the invention.

Indium, zinc, sodium, potassium and iron do not provide any improvement in terms of catalytic and energy performance with respect to the control catalyst without promoter. However, it is observed that the promoters according to the invention allow to improve the catalytic (the conversion rate and the selectivity) and energy performance of the methanation reaction, in particular gadolinium and yttrium.

By using the NiCZ control catalyst without promoter, a maximum of conversion of CO₂ obtained is 73% at 250° C. with an energy consumption of 14.5 W. With the addition of a promoter according to the invention, this conversion rate can be obtained at less power. For example, the addition of gadolinium allows to obtain a conversion of CO₂ of 73-74% at 3.5-3.7 W instead of 14.5 W with the NiCZ catalyst without promoter.

Thus, the catalytic systems according to the invention present the advantage of a gain of 10-12% in the conversion rate with respect to the already existing catalytic systems containing nickel and cerium-zirconium mixed oxide, with a methane selectivity that can reach 100%, at atmospheric pressure and at a temperature between 180 and 230° C., with an energy consumption four times lower.

### II. The Mass Concentration of Promoter

Catalytic systems containing CeZr mixed oxide support with 15% by weight of nickel relative to the weight of the support and yttrium and/or gadolinium as a promoter at various mass concentrations were tested. The mass concentration is expressed in weight of promoter relative to the weight of the support.

The results of their catalytic performance and of the energy consumption according to the mass concentration of the promoter introduced into the catalytic system are summarised in table 2 below.

TABLE 2

| | | | | Rate of | | |
|---|---|---|---|---|---|---|
| | | | Concentration | conversion | Methane | |
| | | | of promoter | of the CO₂ | selectivity | Energy |
| Input | Promoter | Catalyst | (w/w %) | (%) | (%) | performance |
| 0 | — | NiCeZr | 0 | − | ++ | − |
| (control) | | (reference) | | 48 ± 3 | 93 ± 1 | |
| 1 | Y | NiCeZrY-4 | 4 | +++ | +++ | ++ |
| | | | | 76 ± 3 | 97 ± 1 | |
| 2 | Y | NiCeZrY-7 | 7 | +++ | +++ | +++ |
| | | | | 78 ± 3 | 98 ± 1 | |
| 3 | Y | NiCeZrY-10 | 10 | + | ++ | ++ |
| | | | | 56 ± 3 | 95 ± 1 | |
| 4 | Gd | NiCeZrGd-2 | 2 | +++ | +++ | ++ |
| | | | | 76 ± 3 | 97 ± 1 | |
| 5 | Gd | NiCeZrGd-4 | 4 | +++ | +++ | +++ |
| | | | | 83 ± 3 | 98 ± 1 | |
| 6 | Gd | NiCeZrGd-7 | 7 | ++ | +++ | ++ |
| | | | | 73 ± 3 | 95 ± 1 | |
| 7 | Gd | NiCeZrGd-10 | 10 | ++ | ++ | ++ |
| | | | | 70 ± 3 | 94 ± 1 | |
| 8 | Gd | NiCeZrGd-15 | 15 | + | + | + |
| | | | | 59 ± 3 | 92 ± 1 | |
| 9 | Gd and Y | NiCeZrGd-4-Y-7 | Gd-4 and Y-7 | ++ | ++ | ++ |
| | | | | 72 ± 3 | 95 ± 1 | |

The catalytic performance is in particular evaluated according to the conversion rate at a reference power of 8 W. A +++ catalytic performance corresponds to a rate of conversion of the CO₂ greater than or equal to 75%. A ++ catalytic performance corresponds to a rate of conversion of the CO₂ greater than or equal to 60%. A + catalytic performance corresponds to a rate of conversion of the CO₂ greater than or equal to 50%. A − catalytic performance corresponds to a conversion rate lower than 50%.

A +++ methane selectivity corresponds to a methane selectivity greater than or equal to 95%. A ++ methane selectivity corresponds to a methane selectivity greater than or equal to 90%. A − methane selectivity corresponds to a methane selectivity lower than 90%.

The energy performance is evaluated according to the power consumed to obtain a conversion rate of at least 60%. A +++ energy performance corresponds to a consumed power less than or equal to 6 W. A ++ energy performance corresponds to a consumed power less than or equal to 9 W. A + energy performance corresponds to a consumed power less than or equal to 12 W. A − energy performance corresponds to a consumed power greater than 12 W.

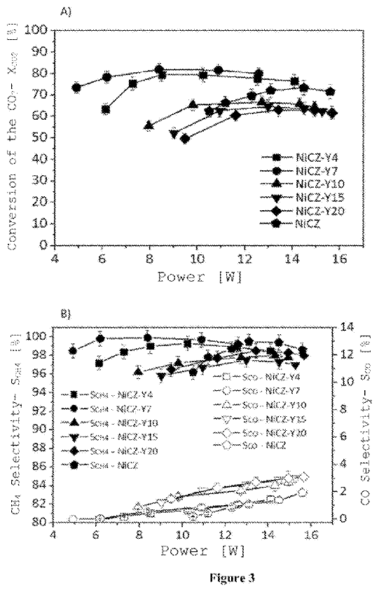

The conversion rate obtained according to the power consumed is shown for each catalyst containing yttrium of table 2 in FIG. 3 (A). As for FIG. 3 (B), it shows the selectivity towards methane with respect to CO according to the power consumed for each catalyst containing yttrium.

At all the mass concentrations studied, and particularly at mass concentrations between 0.1% and 10% by weight relative to the weight of the support, the introduction of the promoter improves the catalytic and energy performance of the reaction with respect to the reference catalyst without promoter. For the catalysts containing gadolinium, the best results were obtained with a mass concentration of 4%. For the catalysts containing yttrium, the best results were obtained with a mass concentration of 7%.

### III. Nature of the Support

Catalysts containing CeZr mixed oxide support, CeO₂ cerium oxide support, or ZrO₂ zirconium oxide support with 15% by weight of nickel and 7% by weight of yttrium as a promoter relative to the weight of the support were tested. The control catalytic systems (inputs 0, 2 and 4) do not comprise promoter.

The results of their catalytic performance and of the energy consumption according to the nature of the support in the catalytic system are summarised in table 3 below.

TABLE 3

| | | | Rate of | | |
|---|---|---|---|---|---|
| | | | conversion | Selectivity | |
| | | | of the | towards | |
| | | | CO₂ | methane | Energy |
| Input | Support | Catalyst | (%) | (%) | performance |
| 1 | CeZr | NiCeZr | + | +++ | − |
| (control) | | (reference) | 62 ± 3 | 97 ± 1 | |
| 2 | CeZr | NiCeZrY-7 | +++ | +++ | +++ |
| | | | 79 ± 3 | 95 ± 1 | |
| 3 | CeO₂ | NiCeO₂ | ++ | ++ | + |
| (control) | | (reference) | 67 ± 3 | 94 ± 1 | |
| 4 | CeO₂ | NiCeO₂Y-7 | ++ | +++ | ++ |
| | | | 72 ± 3 | 95 ± 1 | |
| 5 | ZrO₂ | NiZrO₂ | + | ++ | ++ |
| (control) | | (reference) | 59 ± 3 | 92 ± 1 | |
| 6 | ZrO2 | NiZrO₂Y-7 | ++ | ++ | ++ |
| | | | 68 ± 3 | 94 ± 1 | |

The catalytic performance is in particular evaluated according to the conversion rate at a reference power of A +++ catalytic performance corresponds to a rate of conversion of the CO₂ greater than or equal to 75%. A ++ catalytic performance corresponds to a rate of conversion of the CO₂ greater than or equal to 60%. A + catalytic performance corresponds to a rate of conversion of the CO₂ greater than or equal to 50%. A − catalytic performance corresponds to a conversion rate lower than 50%.

A +++ methane selectivity corresponds to a methane selectivity greater than or equal to 95%. A ++ methane selectivity corresponds to a methane selectivity greater than or equal to 90%. A − methane selectivity corresponds to a methane selectivity lower than 90%.

The energy performance is evaluated according to the power consumed to obtain a conversion rate of at least 60%. A +++ energy performance corresponds to a consumed power less than or equal to 6 W. A ++ energy performance corresponds to a consumed power less than or equal to 9 W. A + energy performance corresponds to a consumed power less than or equal to 12 W. A − energy performance corresponds to a consumed power greater than 12 W.

With the three different supports, the introduction of a promoter allows to improve the catalytic and energy performance of the reaction. The support containing cerium-zirconium mixed oxide allows to obtain the best results.

### IV. The Preparation Process Used

Catalysts containing CeZr mixed oxide support with 15% by weight of nickel and 7% by weight of yttrium or 4% by weight of gadolinium as a promoter relative to the weight of the support obtained via various preparation processes were tested.

The results of their catalytic performance and of the energy consumption according to the nature of the support in the catalytic system are summarised in table 4 below.

TABLE 4

| | | | Rate of | Selec- | |
|---|---|---|---|---|---|
| | | | conversion | tivity | |
| | | | of the | towards | Energy |
| | | Preparation | CO₂ | methane | perfor- |
| Input | Catalyst | process | (%) | (%) | mance |
| 0 | NiCeZr | Wet | − | ++ | − |
| (control) | (reference) | impregnation | 48 ± 3 | 93 ± 1 | |
| 1 | NiCeZr | Coprecipitation | − | ++ | − |
| (control) | (reference) | | 50 ± 3 | 93 ± 1 | |
| 2 | NiCeZrY-7 | Wet | +++ | +++ | +++ |
| | | impregnation | 78 ± 3 | 98 ± 1 | |
| 3 | NiCeZrY-7 | Coprecipitation | ++ | ++ | ++ |
| | | | 73 ± 3 | 95 ± 1 | |
| 4 | NiCeZrY-7 | Sequenced | ++ | ++ | ++ |
| | | impregnation | 69 ± 3 | 95 ± 1 | |
| 5 | NiCeZrGd-4 | Wet | +++ | +++ | +++ |
| | | impregnation | 83 ± 3 | 98 ± 1 | |
| 6 | NiCeZrGd-4 | Coprecipitation | +++ | +++ | +++ |
| | | | 76 ± 3 | 96 ± 1 | |
| 7 | NiCeZrGd-4 | Sequenced | ++ | +++ | +++ |
| | | impregnation | 72 ± 3 | 95 ± 1 | |

The catalytic performance is in particular evaluated according to the conversion rate at a reference power of 8 W. A +++ catalytic performance corresponds to a rate of conversion of the CO₂ greater than or equal to 75%. A ++ catalytic performance corresponds to a rate of conversion of the CO₂ greater than or equal to 60%. A + catalytic performance corresponds to a rate of conversion of the CO₂ greater than or equal to 50%. A − catalytic performance corresponds to a conversion rate lower than 50%.

A +++ methane selectivity corresponds to a methane selectivity greater than or equal to 95%. A ++ methane selectivity corresponds to a methane selectivity greater than or equal to 90%. A − methane selectivity corresponds to a methane selectivity lower than 90%.

The energy performance is evaluated according to the power consumed to obtain a conversion rate of at least 60%. A +++ energy performance corresponds to a consumed power less than or equal to 6 W. A ++ energy performance corresponds to a consumed power less than or equal to 9 W. A + energy performance corresponds to a consumed power less than or equal to 12 W. A − energy performance corresponds to a consumed power greater than 12 W.

Regardless of the preparation process used, the introduction of a promoter allows to improve the catalytic and energy performance with respect to the catalyst without promoter. The process by wet impregnation gives the best results.

### REFERENCES

(1) F. Ocampo, B. Louis, and A.-C. Roger, “Methanation of carbon dioxide over nickel-based Ce_{0.72}Zr_{0.28}O₂ mixed oxide catalysts prepared by sol-gel method”, Applied Catalysis A: General, vol. 369, no 1-2, p. 90-96, November 2009, doi: 10.1016/j.apcata.2009.09.005.
(2) M. Nizio, A. Albarazi, S. Cavadias, J. Amouroux, M. E. Galvez, and P. Da Costa, “Hybrid plasma-catalytic methanation of CO₂ at low temperature over ceria zirconia supported Ni catalysts”, International Journal of Hydrogen Energy, vol. 41, no 27, p. 11584-11592, July 2016, doi: 10.1016/j.ijhydene.2016.02.020.
(3) J. Amouroux and S. Cavadias, “Electrocatalytic reduction of carbon dioxide under plasma DBD process”, J. Phys. D: Appl. Phys., vol. 50, no 46, p. 465501, November 2017, doi: 10.1088/1361-6463/aa8b56.
(4) M. Mikhail et al., “Plasma-catalytic hybrid process for CO₂ methanation: optimization of operation parameters”, Reac Kinet Meeh Cat, vol. 126, no 2, p. 629-643, April 2019, doi: 10.1007/sl 1144-018-1508-8.
(5) M. Mikhail et al., “Electrocatalytic behaviour of CeZrOₓ-supported Ni catalysts in plasma assisted CO₂ methanation”, Catal. Sci. Technol., vol. 10, no 14, p. 4532-4543, 2020, doi: 10.1039/DOCY00312C.
(6) M. Nizio et al., “Low temperature hybrid plasma-catalytic methanation over Ni—Ce—Zr hydrotalcite-derived catalysts”, Catalysis Communications, vol. 83, p. 14-17, August 2016, doi: 10.1016/j.catcom.2016.04.023.
(7) E. Jwa, S. B. Lee, H. W. Lee, and Y. S. Mok, “Plasma-assisted catalytic methanation of CO and CO₂ over Ni-zeolite catalysts”, Fuel Processing Technology, vol. 108, p. 89-93, April 2013, doi:
(8) M. Biset-Peiró, J. Guilera, T. Zhang, J. Arbiol, and T. Andreu, “On the role of ceria in Ni—Al₂O₃ catalyst for CO₂ plasma methanation”, Applied Catalysis A: General, vol. 575, p. 223-229, April 2019, doi:
(9) M. C. Bacariza et al., “DBD plasma-assisted CO₂ methanation using zeolite-based catalysts: Structure composition-reactivity approach and effect of Ce as promoter”, Journal of CO₂ Utilization, vol. 26, p. 202-211, Jul. 2018, doi: 10.1016/j.jcou.2018.05.013.
(10) Amouroux J et al., 2016, European Patent 3050865A1. (11) T. C. Manley, “The Electric Characteristics of the Ozonator Discharge”, Trans. Electrochem. Soc., vol. 84, no 1, p. 83, 1943, doi: 10.1149/1.3071556.
(12) R. Valdivia-Barrientos, J. Pacheco-Sotelo, M. Pacheco-Pacheco, J. S. Benitez-Read, and R. López-Callejas, “Analysis and electrical modelling of a cylindrical DBD configuration at different operating frequencies”, Plasma Sources Sci. Technol., vol. 15, no 2, p. 237-245, May 2006, doi: 10.1088/0963-0252/15/2/008.

What is claimed is:

1. Catalytic system comprising:
a support comprising cerium and/or zirconium,
nickel, and
a promoter chosen from gadolinium, yttrium, strontium, copper, manganese, cobalt, iron and mixtures thereof.

2. Catalytic system according to claim 1, characterized in that the promoter is chosen from gadolinium, yttrium and mixtures thereof, even more preferably from gadolinium and yttrium.

3. Catalytic system according to claim 1, characterized in that the mass concentration of promoter in the catalytic system ranges from 0.1% to 20% by weight relative to the weight of the support, in particular from 0.5% to 15% by weight, preferably ranges from 1% to 10% by weight.

4. Catalytic system according to claim 1, characterized in that the support is a mixed oxide of cerium and of zirconium.

5. Catalytic system according to claim 4, characterized in that the cerium/zirconium molar ratio is within a range going from 90/10 to 40/60, preferably from 80/20 to 50/50.

6. Catalytic system according to claim 1, characterized in that the mass concentration of nickel in the catalytic system ranges from 3% to 30% by weight relative to the weight of the support, preferably ranges from 7% to 17%.

7. Catalytic system according to claim 1, characterized in that the support, the nickel and the promoter form a homogenous mixture.

8. Process for converting a gas comprising CO2 and/or CO in the presence of a catalytic system and a cold plasma, preferably a plasma generated by dielectric barrier discharge (DBD), said catalytic system comprising:
a support comprising cerium and/or zirconium,
nickel, and
a promoter chosen from the lanthanides, yttrium, strontium, copper, manganese, cobalt, iron and mixtures thereof, wherein the lanthanide cannot be cerium.

9. Process according to claim 8, characterized in that it generates:
one or more hydrocarbons, in particular methane, CO, one or more alcohols, formic acid or mixtures thereof, from the conversion of the CO2, and
one or more hydrocarbons, in particular methane, one or more alcohols, formic acid or mixtures thereof, from the conversion of the CO.

10. Process according to claim 8, characterized in that it generates methane.

11. Process according to claim 8, characterized in that the process is carried out in the presence of dihydrogen.

12. Process according to claim 11, characterized in that the molar ratio between the CO2 and/or the CO and the dihydrogen is adapted so as to generate:
one or more hydrocarbons, in particular methane, CO, one or more alcohols, formic acid or mixtures thereof, from the conversion of the CO2, and
one or more hydrocarbons, in particular methane, one or more alcohols, formic acid or mixtures thereof, from the conversion of the CO.

13. Process according to claim 11, characterized in that the process is carried out in the presence of CO2 and the CO2/H2 molar ratio is 1:4, so as to generate methane.

14. Process according to claim 8, characterized in that the catalytic system is as defined in any one of claims 1 to 7.

15. Process for preparing a catalytic system as defined according to claim 1, comprising a step of placing the support or a precursor thereof in contact with a precursor of nickel and a precursor of the promoter, optionally followed by a calcination step.
